(19) United States
(12) Patent Application Publication
(10) Pub. No.: US 2021/0060209 A1
(43) Pub. Date: Mar. 4, 2021

(54) Dura mater biological patch and preparation method thereof

(71) Applicant: Shanghai Baiyiyuan Bioengineering Co., Ltd, Shanghai (CN)

(72) Inventors: Weihong Han, Shanghai (CN); Cuilan Ge, Shanghai (CN); Qiang Qian, Shanghai (CN); Guoqiang Zhang, 201202 (CN)

(21) Appl. No.: 17/003,071

(22) Filed: Aug. 26, 2020

(30) Foreign Application Priority Data
Aug. 29, 2019 (CN) 201910805146.6

(51) Int. Cl. A61L 27/36

(52) U.S. Cl. CPC A61L 27/3645; A61L 27/3629; A61L 2430/40; A61L 27/362; A61L 27/3604; A61L 27/3687

(57) ABSTRACT

The invention of biological dura mater patch and a preparation method thereof. Raw material of patch does not come from commercial meat animals, but from breeding animals, such as SIS patch of the sow, has natural good mechanical properties; the method uses plant-derived reagents in the decellularization process, no damage to ECM natural structural base, and retain more active ingredients, has a better ability to induce tissue repair and growth; neither uses a cross-linking agent to enhance mechanical properties, nor synthetic detergents used to remove cells; chemical residues and their toxicity are avoided, while more effective ingredients in ECM, especially GAGs be retained. The patch has soft texture and good toughness, easy to sew tightly, and prevent cerebrospinal fluid leakage; and degradation rate is basically synchronized with the growth of the new dura mater, which is more conducive to repair and regeneration of dura mater.

### TECHNICAL FIELD

The invention relates to the field of biological materials for tissue repair, in particular to a dura mater biological patch and a preparation method thereof.

### BACKGROUND TECHNIQUE

The dura mater is an important barrier to protect brain tissue, and it is a thick and tough bilayer membrane. The outer layer is the periosteum on the inner surface of the skull, which is only loosely attached to the skull cap, especially when the occipital and temporal parts are more loosely attached, called the periosteum. However, the cranial suture and skull base are more firmly attached and difficult to separate. There is no intradural lumen. The inner layer of the dura mater is thicker and tougher than the outer layer. It is continuous with the dura mater at the occipital foramen. It is called the dura mater. The main function is to protect the brain. Trauma or craniocerebral surgery can cause dura mater defects. The dura mater is used to repair the dura mater and close the subdural space, which can significantly reduce or prevent complications such as cerebrospinal fluid leakage and intracranial infection.

The ideal dura mater should meet the following conditions: 1. It is safe, non-toxic, non-infectious, 2 good tissue compatibility, and no immune rejection. 3 Good density, no permeability, can prevent cerebrospinal fluid leakage and protect brain tissue. 4 It is tough and easy to sew firmly. 5 Can promote the regeneration of dura mater without adhesion. 6 Easy to use, simple operation, easy to disinfect and sterilize. 7 Wide selection of materials and low prices. 8. Has stable biological inertness, does not cause acute and chronic inflammation.

Currently, the most widely used dura mater repair materials are heterogeneous acellular matrixes. This kind of natural biological patch is also called acellular tissue patch. The initial raw material of the patch is mainly derived from the intestinal submucosa, bladder submucosa, and gastric mucosa Inferior layer, pericardium, meninges, amniotic membrane, visceral membrane, peritoneum, dermis and other tissues; through a series of processing, including important processes, such as decellularization, deDNA, α-Gal antigen and other immunogenic components. The acellular matrix material obtained by this method, on the one hand, can retain the three-dimensional structure, but also contains some important active ingredients. The ideal biological patch prepared from acellular matrix should have good biocompatibility, degradability, and absorbability; suitable for mechanical strength, non-toxic, and non-immune; capable of chemotaxis and attachment of host cells, Proliferation and differentiation provide ideal spatial scaffold and suitable micro (nutrition) environment, which is beneficial to the structural repair and functional reconstruction of target tissues.

Regarding the mechanical properties of the patch, pericardium, peritoneum or pleura, tendon, mesentery, etc. derived from commercial meat animals are mostly used as the initial raw material of the dura mater. In practical applications, the mechanical properties of such patches often fail to meet the mechanical requirements of dura mater; therefore, in the process of preparing a dura mater, the following two methods are often used to enhance the mechanical properties of the patch and achieve good toughness, High tensile strength requirements.

One is: chemical methods, in the preparation process of the patch, using chemical crosslinking agents such as epoxide or glutaraldehyde to improve the biomechanical properties of the patch. For example, the patents CN108261565 and CN105999411 use cross-linking agents; the disadvantage is that the degree of cross-linking is difficult to control, the residual cross-linking agent has potential cytotoxicity, and such cross-linking patches usually have good mechanical properties and strong resistance It is not easy to degrade or slow to degrade, and degradation and tissue regeneration cannot be synchronized, which can easily lead to adverse reactions such as fibrosis and chronic inflammation;

The second is: physical methods, such as placing a single-layer sheet horizontally or vertically, and overlapping layers of multiple layers, such as CN106039404A, CN109248339A.

The dura mater products currently on the market are made of bovine tendon extracted from collagen, which is characterized by good biocompatibility and does not require suture; but its degradation rate is too fast, before new tissues are grown, Thedura mater has been degraded prematurely.

Regarding the effective active ingredients in the patch, due to different decellularization methods (mechanical, chemical, enzymatic) and the use of different decellularizing reagents, the effective active ingredients retained in the patch vary greatly; Thomas W. Gilbert et al made a detailed comparison of different decellularization methods and decellularization reagents in Table 1 of the journal “Biomaterials” 27 (2006) No. 3677. In addition to enzymes, acids and bases, detergents (also called surfactants) are used as decellularizing agents, including: Triton X-100, -200, SDS, CHAPS and Betaine type surfactant or Sulfobetaine.

The above detergents are all chemically synthesized or semi-synthetic, with strong detergency and good decellularization effect; but it is easy to cause the loss of effective ingredients such as GAGs in ECM and damage to the natural three-dimensional structure of ECM. The biological patch prepared by using such a decellularization reagent has less effective active ingredients retained and the damage of the three-dimensional structure of ECM is large, so its ability to induce tissue regeneration is poor.

### SUMMARY OF THE INVENTION

The purpose of the present invention is that the existing dura mater biological patch is weak in mechanical strength and cannot effectively meet the clinical practical needs; and the existing method of enhancing the mechanical strength of the dura mater has the aforementioned shortcomings and defects; the inventor Focusing on the specific technical feature of patch biomechanics, through a lot of literature reading, combined with its own careful analysis of academic theory, and many years of rich work experience, the theory and practice are closely combined, which is unexpected but reasonable, clever and convenient To solve the technical problem of insufficient mechanical properties of common dura mater.

In order to achieve the objective of the present invention, on the one hand, the present invention provides a biological patch for dura mater repair, characterized in that the patch contains decellularized connective tissue of breeding stock;

Further, the decellularization decellularization reagent mainly consists of saponin;

Further, the breeding stock is a sow, cow, ewe, and mare;

Further, the breeding stock is a healthy eliminated sow;

Further, the connective tissue refers to one or more combinations of small intestinal submucosa, bladder submucosa, gastric submucosa, dermal matrix, pericardium, meninges, amniotic membrane, organ membrane, and peritoneum;

Further, the connective tissue of the breeding stock refers to the submucosa of the small intestine of the passing sow;

On the other hand, the present invention provides a method for preparing a dura mater biological patch, characterized in that the decellularization reagent in the decellularization process is mainly composed of plant-derived nonionic surfactants;

Further, the decellularization agent is one of plant-derived pentacyclictriterpenesaponins, steroid saponins, or a combination thereof; Further, the decellularization reagent is one of Quil-A, tea saponin, or a combination thereof; Further, the effective working concentration weight ratio of the decellularization reagent is 0.05-1%, the action time with the patch material is 10-60 minutes each time, and the action temperature is 4-15° C.; Further, in the preparation method of the dura mater, the raw material is mainly composed of connective tissue of breeding stock.

The existing dura mater of animal origin is obtained from freshly slaughtered animals and processed through the decellularization process. The fresh slaughtered animal tissues used are commercial hogs, sheep and beef cattle that have just been slaughtered; for example, slaughtered pigs Most of the slaughtered pigs on the farm are commercial pigs that have just been slaughtered. The slaughtered pigs weigh 90-120 kg; the breeding day is 5-6 months.

The inventor solved the technical problem of insufficient mechanical properties of the dura mater. The inventor adopted a completely different technical idea from the existing technology, but started from the first step of preparing the decellularized biological patch, that is, the source of the raw material. Starting from the animal source of this, we will use this as a technical breakthrough point or focus point to carry out in-depth research.

The main innovation of the product of the present invention lies in the selection of animal tissue raw materials to remove cell patches, not the commercial-grade meat animals used by everyone now. Instead, breeding stocks are selected, preferably gilts, and the most preferred is the healthy culled sows due to a decline in reproductive performance; the inventors went deep into the first line of pig breeding, and after fully communicating with the person in charge of a large pig farm, according to their The breeding model and breeding cycle are described. Hogs are slaughtered in 5-6 months and weigh about 90-120 kg; after eight to ten litters, sows are gradually eliminated due to reproductive performance decline. The actual situation for many months, the inventors analyzed and compared the mechanical properties of the two patches prepared from sow SIS and hog SIS, and found that the tensile strength of the sow source SIS patch SIS patches from commercial hogs are significantly higher; at the same time, the tensile strength of SIS patches from eliminated sows is better than the SIS patches from farrowing sows; for comprehensive comparison, the cheaper elimination is preferred Sows are used as raw materials for patches.

The secondary innovation of the product of the present invention is that the preferred tissue site is the porcine small intestinal submucosa (SIS), the second choice is the pig pericardium or peritoneum, and the second choice is the pig dermis and pig bladder; The tablets contain a natural three-dimensional three-dimensional structure, but compared with other tissue parts, the composition of the porcine intestinal submucosa (SIS) is mainly composed of type I fibrous collagen, and also contains type III, IV, and VI collagen. In particular, it contains important type IV collagen, which can obviously promote the formation of new blood vessels and basement membranes; in addition, according to multiple articles published by Dr. STEPHEN F. BADYLAK in the United States, the degradation products of SIS patches have strong antibacterial Active, with functions similar to porcine defensin (pBD-1); and George S. Hussey's 2018 article “Extracellular Matrix Bioscaffolds for Building Gastrointestinal Tissue” reported that SIS patch degradation products also induce cell chemotaxis and The role of mitosis.

The method of the present invention is achieved by using a plant-derived nonionic surfactant to decellularize the key step; the decellularized tissue at the same time is the connective tissue from the breeding stock; the main innovation of the method lies in the preparation of dura mater in the process of tablets, a plant-derived decellularizing agent is used. Such natural nonionic surfactants (such as plant-derived natural saponin and heptylglucoside) can effectively remove cells in the tissue; The cell method is very targeted, mainly by destroying the lipid cell membrane and the membrane of the organelles.

The decellularization effect is not only thorough, but the mode of action is gentle, which does not significantly damage the ECM structure, nor will it cause effective active ingredients in the ECM (such as The obvious loss of glycosaminoglycans, etc.; can retain more effective active ingredients in ECM, including cell growth factors, glycosaminoglycans, etc., which is more conducive to inducing cell chemotaxis, growth, and tissue repair and regeneration; Cells destroy cells, which can be greatly reduced and slowed down. The endogenous enzymes released after the cells are broken may produce some reductions in ECM Damaging effects.

Saponin, a plant-derived decellularizing agent, is a type of secondary metabolite found in certain plants. It is known for producing soap-like foam when oscillated in an aqueous solution; a saponin molecular structure also contains hydrophilic Group and lipophilic group; hydrophilic group is one or more hydrophilic sugar chains, lipophilic group is also called lipophilic mother core, the mother core can be divided into two types namely triterpenes (triterpene) or steroids (Steroids). Commercial saponin products are isolated from the bark of the South American tree species QuillajaSaponariaMolina and the Mexican plant MohaveYucca (also known as Yuccaschidigera), which are derived from seedless. oily plants. The first preferred saponin is QuillajaSaponariaMolina plant extract, also known as Quil-A, CAS No. 8047-15-2, and its critical micelle concentration (CMC, Critical micelle concentration)>0.03%, available from various commercial Purchased through chemical channels, including Sigma, Berghausen Corporation, Sergeant Chemical (Clifton, N.J.), Superfosa/s (Vedbaek, Denmark), and BrenntagBiosector (Frederikssund, Denmark); Quil-A physical and chemical properties can be found in Superfos entitled PurifiedSaponinAdjuvantQuil-A Commercial publications; another preferred saponin is Camellia saponin, referred to as Tea Saponin (Tea Saponin), is a natural surfactant with good performance, more than 90% tea saponin (HPLC grade purity) is easy to prepare and Obtained, such as CN201610990197, the name is: a method for producing high-purity tea saponin; the amount of saponin used as a decellularizing agent in the present invention; the pure saponin is used as the effective component to calculate, the working concentration of decellularization is 0.05%-1%, preferably 0.25-0.5%.

In addition, because the saponin acts in a mild manner, there may be some reversibility of binding to cell membrane lipids; if the subsequent rinsing liquid or soaking liquid has low saponin content or no saponin, it may cause decellularization and debris The effect is reduced; therefore, the solution containing saponin should also be used in the next washing solution or bubble liquid after decellularization; the original concentration of saponin solution can be selected to be washable or soaked to facilitate the more complete removal of cells and their cells Debris.

### PRINCIPLE OF INVENTION

In the patch product of the present invention, the source of the patch raw material is breeding stock. For example, a sow that has had several litters has a feeding duration of more than 24 months; a normal sow produces 2-2.3 litters per year, and the average sow gestation period is 114 days, empty period is 10-15 days, lactation days are 21-28 days; culled sows is mainly due to decreased reproductive performance, such as fewer litters, more weak litters, difficult to breed or less milk, weaning litter weight Due to light factors, sows are usually eliminated, and their breeding months reach more than 40 months. Compared with commercial hogs, all aspects of tissue and organ development of sows are completely developed. At the sub-organ level and structure, Observation and microscopic performance, histological strength and other indicators have achieved true and full and comprehensive maturity; further from the molecular level and molecular structure, it is possible that the degree of collagen hydroxylation is high, the stability of the triple helix structure is better, and collagen The natural cross-linking degree is high and the thermal stability is good; macroscopically, the mechanical properties of the connective tissue are better.

The prepared patch has high tensile strength. Now the sources of patch raw materials are all commercial meat animals such as hogs, which usually have a short feeding period of only 5-6 months, and their slaughter weight is 90-120 kg, which is relative to the sows (including the culled sows). 180-200 kg) in terms of body weight and size, the weight of commercial hogs is obviously lighter, and their volume is smaller; and when commercial hogs are slaughtered, their breeding duration is only 5-6 Months, and the breeding period of sows is at least 12 months (based on only one litter), and the culled sows will reach 40 months; the breeding time is 2 to 8 times that of commercial pigs; At the same time, the tissues and organs of commercial hogs, compared with sows, are only beginning to take shape on the surface and the frame structure is shaped. However, in fact, their internal structure, microscopic performance, histological strength and other indicators are not really thorough and complete.

Fully mature, further from the molecular level and molecular structure, it is possible that the degree of collagen hydroxylation is low, the stability of the triple helix structure is poor, the degree of natural cross-linking between collagens is low, and the thermal stability is poor; macroscopically, it is expressed as connective tissue Mechanical properties Difference to prepare a patch, detected low tensile strength.

In the method of the present invention, the decellularizing agent is selected from plant-derived nonionic surfactants, preferably plant-derived saponins. The decellularization of such agents is highly targeted, mainly by destroying lipid cell membranes and organelle membranes. Strong, no damage to ECM structure, will not cause the loss of effective components (such as glycosaminoglycans) in ECM; compared with other chemical or semi-synthetic detergents, the decellularization effect is complete, but The plant-derived saponin has a unique and gentle mode of action. Using this type of plant-derived saponin for decellularization can retain more effective ingredients in ECM; such as the patch prepared by the method of the present invention, and the chemical-based detergent decellularized preparation Compared with tablets, the glycosaminoglycan content in its patch is significantly higher; the glycosaminoglycan sugar chain structure has a high complexity and spatio-temporal specificity, which is the difference of sugar chain synthesis related enzymes in different cell tissues and organs Due to different expression control methods and levels at the developmental stage, the complexity of the glycosaminoglycan structure gives it functional diversity and adaptability.

Compared with the prior art, the present invention has the following significant advantages and beneficial effects:

1. The dura mater patch product of the present invention uses breeding animal tissue as raw material instead of commercial meat and livestock, has better natural cross-linking degree, the patch has good toughness, high tensile strength, is not easy to break, and can prevent cerebrospinal fluid leakage.

2. The dura mater patch product of the present invention uses the submucosa of the small intestine as a raw material, retains the three-dimensional structure of the extracellular matrix, various functional proteins, growth factors, glycosaminoglycans and other components, and has a good function of inducing tissue regeneration. It can accelerate the growth and functional reconstruction of postoperative dural tissue.

3. The product of the present invention has no cross-linking agent and synthetic detergent residues, does not have potential cytotoxicity, and does not cause fibrosis or chronic inflammation.

4. The dura mater product of the present invention has an ECM three-dimensional structure that induces cell and blood vessel ingrowth, and gradually degrades itself as new tissue grows. The degradation product peptide component has antibacterial properties and can reduce inflammation after implantation. And the occurrence of infection.

5. In the method of the present invention, the use of plant-derived nonionic surfactants as decellularization agents, instead of chemical detergents, will not cause a large loss of effective ingredients in ECM.

In addition, economically speaking, the raw material can be the sows that have been eliminated due to the decline in reproductive performance. Generally, the price of such raw materials is significantly lower than that of commercial hogs; for the future large-scale purchase of raw materials, the eliminated sows are used as The raw material for the decellularization of connective tissue can save enterprises a lot of expenses, which is also one of the innovations.

The purpose of the present invention is to provide a dura mater and its preparation method. In order to achieve the purpose of the present invention, it is specifically achieved by the following technical solutions.

For the decellularized dura mater, the animal tissue raw materials are derived from adult breeding stocks, rather than commercial meat stocks.

Further, the adult breeding stock includes breeding pigs, breeding cattle, breeding sheep, stallions and the like.

Further, it is preferred that the adult breeding stock is a sow, cow, ewe, mare, and the like.

Further, it is preferable that the adult breeding stock is a sow, cow, ewe, mare, etc., which have been eliminated due to a decrease in reproductive performance.

Further, the raw materials of the decellularized dura mater include one or more raw materials of small intestinal submucosa, bladder submucosa, gastric submucosa, dermal matrix, pericardium, meninges, amniotic membrane, organ membrane, and peritoneum combination.

Further, the preferred animal tissue raw material is the small intestinal submucosa of eliminated sows.

Further, the decellularized dura mater patch is obtained by washing, disinfecting, defatting, decellularizing, decellularizing, removing DNA and removing α-Gal antigen, finalizing, lyophilizing and sterilizing.

The invention also provides a method for preparing a dura mater, which includes:

Step 1. Material and washing: take the small intestine of breeding stock and wash it fully;

Step 2. Pretreatment: mechanical scraping to remove the mucosal layer, muscular layer, serous membrane layer, and lymph nodes of the small intestine to separate the submucosa; rinse the submucosa of the small intestine, soak in acetic acid solution, soak time 30-120 minutes, The ratio of the intestinal submucosa to the acetic acid solution is 1:5-1:10; the raw material for the pre-treatment patch is obtained;

Step 3. Pre-sterilization: use a mixed solution containing peroxyacetic acid and ethanol to soak the raw materials of the patch under ultrasound and room temperature conditions for disinfection; the concentration of peroxyacetic acid is 0.5-1.5% and the concentration of ethanol is 15-25%. The ratio of the raw material of the tablets to the mixed aqueous solution is 1:5-1:10, and the soaking time is 30-120 minutes; then it is ultrasonically cleaned with purified water;

Step 4. Degreasing: use ethanol solution, soak the raw material of the patch under ultrasound and normal temperature, the concentration of ethanol is 90-100%, the ratio of the raw material of the patch and ethanol is 1:5-1:10, and the soaking time at normal temperature is 0.5-6 h; afterwards, ultrasonic cleaning with water for injection;

Step 5. Decellularization: use a solution containing plant-derived saponin, soak the raw materials of the patch at 4-15° C. and ultrasound for 10 to 60 minutes; the ratio of the raw materials of the patch to the solution is 1:10 (W/V); Soak the fresh saponin solution to the raw material of the patch for 5-60 minutes; then soak in PBS-EDTA for 10-60 minutes; repeat the decellularization 1-3 times;

Step 6. To remove DNA and to remove α-Gal antigen: soak the raw material of the patch with an aqueous solution containing DNAse, the soaking temperature is 36 and the soaking time is 15-40 minutes; after washing, use the aqueous solution containing α-galactosidase, Soak the raw materials of the patch, the soaking time is 15-40 minutes;

Step 7. Use 10 mM NaOH aqueous solution, soak the raw material of the patch under normal temperature and ultrasonic conditions; then use ultrasonic cleaning with PBS until neutral;

Step 8. The semi-finished patch is made into a sheet shape, fixed on the mold by overlapping and overlapping, freeze-dried, and the hard film patch can be obtained by packaging and irradiation sterilization.

Further, in the decellularization process (step 5), the following technical parameters are preferred:

The content of effective saponin in saponin solution is 0.05-1% (W/W), the ratio of patch raw material to plant-derived saponin solution is 1:5-1:10, under ultrasonic conditions, in Soak at low temperature 4-10 for 20-45 minutes; then soak the fresh patch material with fresh saponin solution of the same concentration for 5-30 minutes; then soak in PBS-EDTA for 10-30 minutes; repeat Cell 1 time.

Further, in the decellularization process (step 4), more preferably, the following technical parameters:

The content of effective saponin in the saponin solution is 0.25-0.5% (W/W), the ratio of the patch material to the plant-derived saponin solution is 1:10, and soaked under ultrasonic conditions at a low temperature of 4° C. for 20 minute;

Further, the plant-derived surfactant refers to one or a combination of plant-derived triterpenesaponins and steroid saponins;

Further, the plant-derived triterpenesaponin is one of Quil-A source, tea saponin, or a combination thereof;

Further, the working concentration of saponin is 0.25-0.5% (W/W). Note that the effective working concentration is not calculated according to the commodity.

Further, the immersion time required for saponin solution to remove cells in tissue is 20-30 minutes; the working/action temperature is 4° C.

The terms preferably understood according to the following text descriptions; other terms are understood according to the level of those of ordinary skill in the art.

### EXPLANATION OF TERMS AND TERMS

1. Breeding livestock: for commercial meat animals; the main purpose of breeding is to breed young animals, not short-term breeding as commercial meat animals; the main purpose of raising commercial meat animals is to grow meat for Human consumption to provide protein of animal origin; the breeding animals in this patent include pigs, cows, sheep, horses, donkeys, camels, and dogs; the breeding animals are all adult and capable of reproduction, usually for at least one year Above; for example, breeding pigs include sows and boars, where sows refer to adult sows that can be bred, excluding young gilts.
2. Born female: a part of a breeding stock that is a female that has at least one birth; it is relative to a backup female, which mainly refers to the one who has not yet reached pregnancy because of the age or the age of the month. Young female.
3. Culled matured females livestocks: originally part of the breeding stock; in the farm, the manager considered that when the females' reproductive performance dropped to a certain level, such as fewer litters, low milk production, light weaning litters, and comprehensive consideration of the female pen Various factors such as the utilization rate of the sows and the price of the sows are eliminated; judging and analyzing from the perspective of economics, it is believed that the value obtained by continuing to raise the sows has been significantly reduced. There are some differences in the judging criteria; for example, sows are gradually eliminated after giving birth to 8 litters; there is a slight difference between pigs of different breeds.

The following further describes the principles and solutions of the present invention in conjunction with specific embodiments; it should be understood that these embodiments are only for illustration and to facilitate understanding of the ideas of the present invention, but cannot be limited thereto; the embodiments do not limit the present invention in any way Scope, in the following embodiments, various processes and methods not described in detail are conventional methods well known in the art.

### DETAILED DESCRIPTION

### Example 1

The specific steps for the preparation of porcine small intestinal submucosa dura mater are as follows:

1) Material extraction and washing: pre-treatment: according to the breeding records, select the breeding days of about 24 months, the binary mixed sow in the empty stage, after slaughtering, clean the fresh small intestine tissue;

2) Pretreatment: remove the mucosal layer, muscular layer, serous membrane layer, and lymph nodes of the pig small intestine by physical scraping, separate the submucosa, and soak in 0.5% acetic acid solution for 30 minutes. The ratio of pig small intestine to acetic acid solution is 1:5. Soak in purified water three times to obtain the raw material of the biological patch, namely the submucosa of the small intestine, hereinafter referred to as SIS material;

3) Sterilization: Use a mixed aqueous solution containing 1.0% peroxyacetic acid and 15% ethanol. The ratio of SIS material to mixed aqueous solution is 1:10. Under ultrasonic conditions, immerse at room temperature for 100 minutes for disinfection. After that, use purified water for ultrasonic cleaning 3 times;

4) Degreasing: use ethanol with a concentration of 90%, the ratio of SIS material to ethanol is 1:10, under ultrasonic conditions, soak at room temperature for 2 h; afterwards, use ultrasonic cleaning with water for injection 3 limes;

5) Decellularization: use a solution containing 0.25% saponin (from Quil-A, the working concentration is calculated based on the content of pure saponin), soak the raw material of the patch at 4° C. and ultrasonic conditions for 30 minutes; then use the same concentration of 0.5 The saponin solution is used to rinse the patch material for 10 minutes; then the patch is soaked with PBS-EDTA solution for 20 minutes; the previous decellularization step is repeated once, and the total time is about 120 minutes;

6) Remove DNA and remove α-Gal antigen: use an aqueous solution containing SU/ml DNase, the ratio of SIS material to DNase solution is 1:5, soak for 20 minutes at 37° C. under ultrasound; then use PBS Rinse 3 times; use an aqueous solution containing SU/ml α-galactosidase, the ratio of SIS material to α-galactosidase solution is 1:5, soak for 20 minutes at 30° C. under ultrasound; then use PBS solution rinse;

7) Use an aqueous solution of NaOH with a concentration of 10 mM, the ratio of SIS material to NaOH solution is 1:20, under ultrasonic conditions, soak for 50 minutes at room temperature; then use PBS ultrasonic cleaning until neutral;

8) Stereotypes, freeze-drying and sterilization: the decellularized sheet-like raw materials are intersected horizontally and crosswise among the four pieces, and are fixed on the mold overlappingly. After freeze-drying, packaging, and finally irradiation sterilization.

### Example 2

The steps of pretreatment of small intestine tissue, disinfection, defatting, decellularization, deDNA removal and α-Gal antigen removal, lyophilization, sterilization, etc., are completely the same as in Example 1; the difference is only in the initial animal selection, in slaughter In the field, the fresh small intestine of the eliminated sow is selected as the raw material for the patch; after in-depth understanding, the eliminated binary sow usually has a gestational age of more than 8 births and weighs about 180 kg, which is empty; after retrospective investigation and understanding, the elimination Sows have been raised for more than 40 months.

### Example 3

The steps of pretreatment of small intestinal tissue, disinfection, defatting, decellularization, deDNA and α-Gal antigen removal, lyophilization, sterilization, etc. are same as in Example 1; the difference is only in the initial animal selection; the slaughter is selected On the farm, the fresh small intestine tissues of DuchangSanyuan groceries pigs weighing about 100 kg were washed and cleaned; it is known that the number of breeding days is 160-180 days.

### Example 4

Animal selection, pretreatment of connective tissue, disinfection, defatting, decellularization, deDNA removal and α-Gal antigen removal, lyophilization, sterilization and other steps are exactly the same as in Example 3; the difference is only the choice of decellularization reagent in the fourth step Above, in this embodiment, 0.25% SDS was used to replace 0.25% saponin in Embodiment 3.

### Example 5

Animal selection, connective tissue pretreatment, disinfection, defatting, decellularization, deDNA removal and α-Gal antigen removal, lyophilization, sterilization and other steps are same as in Example 1; the difference is only in the selection of decellularization reagents in the fourth step in this example, 0.5% tea saponin was used instead of 0.25% saponin in Example 1.

### Example 6

Animal selection, connective tissue pretreatment, disinfection, defatting, decellularization, deDNA removal and α-Gal antigen removal, lyophilization, sterilization and other steps are same as in Example 2; the difference is only in the selection of decellularization reagents, In this example, 0.5% teasaponin was used instead of 0.25% saponin in Example 1.

### Example 7: Optical Observation and Effective Component Detection of Dura Mater

Optical Microscope Observation:

Method: fixed with formalin, embedded in paraffin, cut the patch in the example into thin slices, dewaxed with xylene, dehydrated with alcohol, stained with hematoxylin-eosin, and observed the residual cells and matrix fibers under the microscope structure.

Results: In all of the decellularized patches in the six examples, no cells and fragments were observed; collagen fibers were continuous and of varying thickness, but no obvious breakage was observed.

Quantitative detection methods of glycosaminoglycans (GAGs) include Dubious method, Carbazole method, Elson-Morgan method, ELISA and electrophoresis method.

For the patch samples prepared in 1-6 examples, the content of the important active component glycosaminoglycans (GAGs) was detected using a commercial ELISA kit; the pretreatment method was to use low-temperature grinding method for various patches After processing, the results are shown in Table 1:

| | | Glycosaminoglycan |
|---|---|---|
| Group | Patch source and treatment methods | GAGs (ug/mg) |
| Example 1 | Sow's SIS + 0.25% saponin | 6.43 ± 0.38 |
| Example 2 | Culled sowSIS + 0.25% saponin | 5.85 ± 0.63 |
| Example 3 | Commercial Hog SIS + 0.25% saponin | 6.56 ± 0.56 |
| Example 4 | Commodity Hog SIS + 0.25% SDS | 3.65 ± 0.67 |
| Example 5 | Sow'sSIS + 0.5% tea saponin | 6.20 ± 0.39 |
| Example 6 | Culled sow SIS + 0.5% tea saponin | 5.68 ± 0.54 |

### Example 8: Detection of Mechanical Properties of Dura Mater

The tensile strength of the dura mater samples prepared in Examples 1-6 was tested.

Method: Cut the sample into a shape with a width of 10 mm in two directions; after cutting, the sample was placed in an environment with a relative humidity of 40%-60% and a temperature of 22±2° C. for 2 hours. The distance between the clamps is 25 mm. Fix the two ends of the sample on the chuck of the tensile tester and stretch at a speed of 100 mm/min. Record the maximum force value at break.

The results are shown in Table 2 below:

| | | Toughness |
|---|---|---|
| | | (tensile strength) |
| Group | Patch source and treatment methods | Unit: N |
| Example 1 | Sow's SIS + 0.25% saponin | 48 |
| Example 2 | Culled sow SIS + 0.25% saponin | 53 |
| Example 3 | Commercial Hog SIS + 0.25% saponin | 34 |
| Example 4 | Commodity Hog SIS + 0.25% SDS | 29 |
| Example 5 | Sow's SIS + 0.5% tea saponin | 48 |
| Example 6 | Culled sow SIS + 0.5% tea saponin | 50 |

A person of ordinary skill in the art can make various simple changes, adjustments or combinations to the present invention according to the above description; therefore, without prejudice to the spirit of the claims of the present invention, certain details in the embodiments should not it constitutes a limitation to the present invention, and the present invention will take the scope defined by the appended claims as the protection scope.

1. A biological patch for repairing dura mater, wherein the patch contains decellularized connective tissue of breeding stock, the breeding stock is specifically adult, reproducible stock, kept for at least one year, excluding young reserve breeding stock, as contrasted to commercial meat stock.

2. The patch of claim 1, wherein the decellularizing agent is mainly composed of saponin.

3. The patch of claim 1, wherein the breeding stock includes sow, cow, ewe, and mare.

4. The patch of claim 1, wherein the connective tissue is one of, or a combination of any of: the submucosa of the small intestine, the submucosa of the bladder, the submucosa of the stomach, the dermal matrix, the pericardium, the meninges, the amniotic membrane, the visceral membrane, and the peritoneum.

5. The patch according to claim 1, wherein the connective tissue of the breeding stock is the submucosa of the small intestine of the sow.

6. A method for preparing the patch of claim 1, wherein the decellularization reagent in the decellularization process is mainly composed of plant-derived nonionic surfactant.

7. The method according to claim 6, wherein the decellularizing agent is one of plant-derived triterpenesaponins, steroid saponins, or combination thereof.

8. The method according to claim 6, wherein the decellularizing agent is one of Quil-A, tea saponin, or combination thereof.

9. The method according to claim 6, wherein the effective working concentration weight ratio of the decellularizing agent is 0.05-1%; the decellularizing agent acts on the patch multiple times, each action time being the action time with the patch material is 10-60 minutes, and action temperature being 4-15° C.

10. The method according to claim 6, wherein raw material of the method for preparing the dura mater is mainly composed of connective tissue of the breeding stock.
